(19) United States
(12) Patent Application Publication
(10) Pub. No.: US 2008/0262458 A1
(43) Pub. Date: Oct. 23, 2008

(54) Absorbent Article With Wiping Function

(75) Inventor: Pontus Winqvist, Stora Hoga (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 11/596,457

(22) PCT Filed: May 13, 2004
(86) PCT No.: PCT/SE2004/000734

§ 371 Date: Jan. 10, 2007

(51) Int. Cl. A61F 13/53

(52) U.S. Cl. 604/374; 604/378

(57) ABSTRACT

An absorbent article (101; 201), such as a baby diaper (101) or an incontinence guard (201), in which the rear part of the absorbent article's liquid-permeable cover sheet (102; 202) oriented towards the body includes a receiving area (135; 235) for faeces. The article further includes a wiping area (131; 231) arranged outside the receiving area (135; 235), said wiping area (131; 231) being characterized in that it includes a separate material section (132; 232) including a wiping material (136; 236) intended for wiping faeces from a user dirtied with faeces.

### TECHNICAL FIELD

The invention relates to an absorbent article such as a baby diaper or an incontinence guard. The article comprises a liquid-permeable cover sheet intended to be directed towards the user during use, and a backing sheet intended to be directed away from the user during use. An absorbent body is arranged between the liquid-permeable cover sheet and the backing sheet. The article further comprises a rear transverse edge and a front transverse edge, the distance between the rear transverse edge and the front transverse edge constituting the total length of the article. The absorbent article comprises a receiving area for faeces arranged on the liquid-permeable cover sheet, said receiving area being arranged between a transverse line, located at a distance of 60% of the total length of the article away from the rear transverse edge, and the rear transverse edge of the absorbent article. The absorbent article also comprises a wiping area intended for removing faeces, said wiping area being arranged outside the receiving area for faeces.

### BACKGROUND

Baby diapers and incontinence guards are absorbent articles which are intended to cover the lower part of a user's trunk and which comprise a crotch portion and also a front waist portion and a rear waist portion.

The changing of both baby diapers and incontinence guards is in principle always carried out by a care provider, such as one of the child's parents in the case of baby diapers, and by an employed career or relative in the case of an incontinence guard.

When changing absorbent articles as above, it is not uncommon for the lower abdomen of the user to be heavily soiled by faeces, in which case the lower abdomen has to be thoroughly cleaned before a new article can be fitted on the user.

It is customary for the faeces to be removed in several stages, the first stage being a rough cleaning of the soiled lower abdomen with the aim of removing the greater part of the faeces clinging to the user. Thereafter, in a second cleaning stage, the soiled lower abdomen is cleaned or washed more thoroughly with the aim of preparing the child or incontinent person for application of a new absorbent article.

In U.S. Pat. No. 5,607,760, the problem of cleaning a user's lower abdomen, soiled with faeces, is tackled by providing that surface of the article facing the body with a special lotion which, at the time of use, is transferred to the user's skin. The transferred lotion reduces the tendency of the faeces to stick to the wearer's skin, thus making removal of the faeces easier.

Another approach aimed at making it easier to clean a user's lower abdomen has been to provide absorbent articles with special receiving areas for faeces.

The objective in designing these articles has been to provide the articles with properties which mean that a greater amount of the excreted faeces clings to the article and a lesser amount to the wearer's skin. A common feature of most of these articles is that the receiving areas for faeces, that is to say the rear parts of the articles, have been provided with different types of cover sheets specially adapted for receiving and storing faeces.

U.S. Pat. No. 6,648,865 is concerned with separating low-viscosity faeces from more viscous faeces, which of course reduces the problem of the skin of a user's lower abdomen becoming soiled with faeces.

Patent application WO 00/28929 describes an absorbent article comprising a special receiving area for faeces arranged in the rear part of the article, the special receiving part comprising a receiving material which has relatively large openings through which faeces can penetrate into the article, and in which openings the faeces easily attaches. The main problem which the application seeks to solve is that of ensuring that faeces can pass through the uppermost cover sheet of the article and be concealed in more absorbent layers of material lying underneath.

In one embodiment, the article has an integrated cover sheet, with openings having been formed in the zone of the cover sheet for receiving faeces, and other parts of the article's cover sheet consist of the same cover sheet but without any special openings.

it is further described how the zone comprising openings can be treated with hydrophobic means, or, alternatively, the area without openings can be treated with hydrophilic means. The choice of hydrophobic means or hydrophilic means depends on the degree of hydrophobicity/hydrophilicity the cover sheet has before treatment. Irrespective of which treatment is carried out, the desired final result is a comparatively, hydrophobic receiving zone for faeces and a comparatively hydrophilic receiving zone for urine. It is also stated that the hydrophilic part of the cover sheet, at the front parts of the article, is above all optimized for rapid passage of urine, but it is also stated that it can also function as a first wiping area for removing faeces from the lower abdomen of a user.

Irrespective of the effectiveness of the described absorbent articles as regards handling of faeces, there is therefore a need for an article which is improved in terms of the removal of faeces from a user's lower abdomen when the article is being changed.

It is customary for faeces to be removed from a soiled lower abdomen in several stages, the first stage being a rough cleaning of the soiled lower abdomen with the aim of removing the greater part of the faeces clinging to the user. A subsequent more thorough cleaning or washing of the lower abdomen is then carried out in a second stage.

The initial wiping/rough cleaning can be done in a number of different ways, it being possible to choose between a number of different wiping aids.

Many parents and care providers use the diaper or incontinence guard, which has just been removed from the user, for the initial wiping. The diaper or incontinence guard is usually gripped on the side facing away from the user, the diaper or incontinence guard being crumpled up so that a good grip is obtained. The initial wiping is then carried out using the crumpled-up diaper or incontinence guard. The aim of this initial wiping is to remove the majority of the faeces located on the user's skin. Another aim of the wiping operation is to collect as much as possible of the faeces in the article before the latter is disposed of. The absorbent article, containing both urine and faeces, is then folded or rolled up and closed in a suitable way before being discarded.

The care provider normally tries to carry out the wiping operation using a suitable part of the diaper or incontinence guard which is not soiled with faeces when the wiping operation is started.

An advantage of the described wiping method is that no extra wiping material is used up for the initial rough cleaning of the user's lower abdomen, which means a gain both in financial and environmental terms.

A disadvantage of using the article for this initial cleaning stage is, however, that the absorbent article is not especially well suited as a wiping aid.

Another common way of solving the problem of removing faeces from a user's skin is of course to use a special wiping paper, for example conventional toilet paper, for the initial rough clean.

Various types of wiping materials intended for faeces, for example, are described in several different patent documents. U.S. Pat. No. 6,420,013, to give one example, describes a surface structure and chemical additives used for a soft and efficient wiping material intended for faeces.

Unnecessarily large amounts of wiping material/toilet paper are usually used up in this way of removing faeces from the user's lower abdomen.

Special wash cloths are also used by many people for both the initial rough wiping and also for the subsequent and more thorough cleaning of the user's lower abdomen.

Specially designed washing mittens for cleaning the soiled lower abdomen of users of diapers or incontinence guards are also available on the market. A washing mitten normally consists of two basically rectangular material sheets which have been interconnected along three of their edges so that a pocket is formed between the material sheets. When using the washing mitten, the hand of the person carrying out the wiping is placed inside the pocket of the washing mitten, after which wiping is carried out. The hand is then well protected against soiling.

An example of a washing mitten of the type indicated above is described in patent document WO 96/16217.

The main disadvantage of all these different types of separate wiping aids, for example toilet paper, wash cloths or special washing mittens, is that it takes a certain time to get hold of them when wiping is to be carried out. For example, toilet paper has to be unwound from the toiler paper roll, the wash cloth has to be removed from its package, and so on. Often, the separate wiping article is not even located at a place where it can be quickly found, so that a certain amount of time is lost in looking for the wiping article. During the short period of time from the point where the diaper or incontinence guard is opened to when the wiping operation is carried out, the baby or the incontinent person may move and in so doing get excrement smeared on his or her skin and on surroundings. In addition, when seeking the wiping aid, the care provider or parent also often has to keep an eye on the baby or incontinent person. Babies in particular, because of their suppleness, may end up getting their feet or hands in the excrement.

Another disadvantage of toilet paper, wash cloths and special washing mittens is that the wiping material, regardless of which type it is, is used only during the very short time cleaning is carried out. Immediately after the cleaning has ended, the toilet paper, wash cloth or washing mitten is discarded and then constitutes an inconvenient and problematic waste component, which further adds to the amount of refuse without providing any additional benefit. There may also be problems in handling large amounts of faeces with the aid of, for example, a wash cloth or washing mitten.

The cost of these various wiping articles is also felt by many to be extremely high in relation to the limited benefit which use of washing mittens, wash cloths or toilet paper brings. The wastefulness involved in using separate wiping articles is also a source of annoyance to many parents and care providers.

### OBJECTS AND SUMMARY

There is therefore a need for an effective article for initial wiping/removal of faeces from the soiled lower abdomen of a user of diapers or incontinence guards, and which article is available immediately after the diaper or incontinence guard has been detached from the user.

There is a further need for an article for initial wiping/removal of faeces from the soiled lower abdomen of a user of diapers or incontinence guards, which article is not perceived as being expensive and which is not regarded as involving an unnecessary waste of resources.

By means of the present invention, an absorbent article of the type referred to in the introduction has now been produced, which absorbent article eliminates the problems associated with previously known such absorbent articles.

An absorbent article designed according to the invention is characterized mainly in that the wiping area comprises a separate material section connected to the absorbent article, the separate material section comprising a wiping material intended for wiping faeces from a user dirtied with faeces.

According to a preferred embodiment, the material section constituting the wiping material is arranged on the liquid-permeable cover sheet of the absorbent article.

According to one embodiment, the absorbent article is a diaper intended for infants. The material section constituting the wiping material in this case has an extent, in the longitudinal direction of the diaper, of 10-40% of the total length of the diaper, and the material section preferably has an extent of 25-35% of the total length of the diaper.

According to a further embodiment, the absorbent article is a diaper intended for infants, in which the material section constituting the wiping material has an extent, in the transverse direction of the diaper, of at least 12 centimetres, preferably at least 15 centimetres.

According to a further embodiment in which the absorbent article is a diaper intended for infants, the material section constituting the wiping material has an extent across the full width of the diaper in the transverse direction of said diaper.

According to one embodiment, the absorbent article is an incontinence guard intended for adults. The material section constituting the wiping material in this case has an extent, in the longitudinal direction of the incontinence guard, of 20-30% of the total length of the incontinence guard, and the material section preferably has an extent of 25% of the total length of the incontinence guard.

According to a further embodiment, the absorbent article is an incontinence guard intended for adults, in which the material section constituting the wiping material has an extent, in the transverse direction of the diaper, of at least 15 centimetres, preferably at least 18 centimetres.

According to a further embodiment in which the absorbent article is an incontinence guard intended for adults, the material section constituting the wiping material has an extent across the full width of the incontinence guard in the transverse direction of said incontinence guard.

According to a preferred embodiment, the wiping material comprises cellulose fibres.

According to another embodiment, the wiping material comprises rayon fibres.

According to one embodiment, the wiping material on the absorbent article comprises means which increase the dry strength of the wiping material and/or means which increase the wet strength of the wiping material.

According to one embodiment of the absorbent article, the wiping material comprises means which increase its softness.

According to one embodiment of the absorbent article, the wiping material has a folded three-dimensional structure.

According to one embodiment, the folding has a frequency of 1-20 cycles/centimetre, preferably 2-12 cycles/centimetre, and most preferably 3-8 cycles/centimetre.

According to one embodiment of the absorbent article, the wiping material has a structure comprising openings.

According to one embodiment, the openings have a frequency of 1-20 openings/centimetre, preferably 2-12 openings/centimetre, and most preferably 3-8 openings/centimetre.

According to one embodiment of the invention, the absorbent article is a pair of diaper pants, and according to another embodiment it is a belt diaper.

In one embodiment, the material section constituting the wiping material is arranged on the backing sheet of the article.

### DESCRIPTION OF THE FIGURES

The invention will be described in greater detail below with reference to the figures shown in the accompanying drawings, in which:

FIG. 1 shows a disposable diaper intended for infants, according to a first embodiment of the invention.

FIG. 2 shows an incontinence guard intended for incontinent adult users, according to a second embodiment of the invention.

### DESCRIPTION OF ILLUSTRATIVE EMBODIMENTS

One embodiment of the invention relates to absorbent articles such as baby diapers, incontinence guards or the like. The diapers or incontinence guards can be in the form of so-called open diapers or incontinence guards which are joined round the waist of a user when fitted. Alternatively, the diapers or incontinence guards can be so-called diaper pants, in which case they are designed as complete pants with a joined-up waist area. Diaper pants are fitted by pulling them up over the user's legs exactly like a normal pair of pants. When a diaper or an incontinence guard of the pants type is removed from the user, it is customary for the joined-up waist area to be torn or cut apart. Another conceivable type of diaper is a belt diaper. Such a diaper is fixed to a belt which is fitted round the user's waist. The belt can be a separate belt which has been fixed round the user, or it can be a belt which is integrated with the diaper or incontinence guard.

Another embodiment according to the invention can also be a so-called insert diaper or an incontinence guard intended to be fixed with the aid of a securing diaper. The diaper or the incontinence guard in this case does not have its own securing members, and instead it is inserted into underpants or into special securing pants at the time of use.

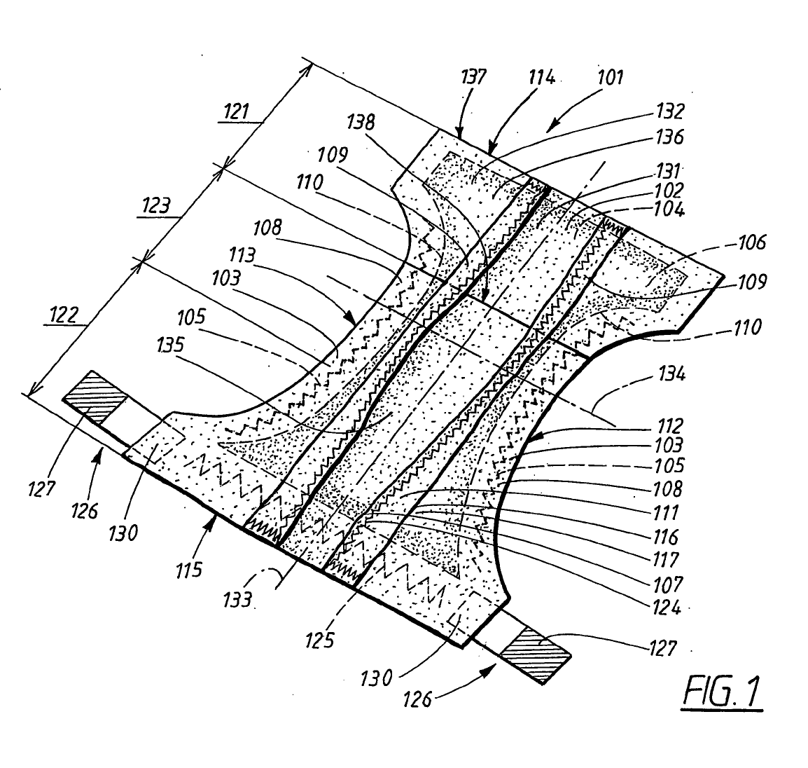

The first embodiment shown in FIG. 1 concerns a baby diaper 101 shown from that side of the diaper 101 which, during use, is intended to face towards the user. The diaper 101 in FIG. 1 is shown stretched flat out.

The diaper 101 is essentially hourglass-shaped and has longitudinal edges 112,113, a front transverse edge 114 and a rear transverse edge 115. The diaper 101 also has a front end portion 121, a rear end portion 122 and a narrower crotch portion 123 located between the end portions 121, 122. During use, the crotch portion 123 is intended to be located at the narrowest area between the thighs of the user.

A longitudinal line of symmetry 133 which divides the article into a right-hand half and a left-hand half is indicated in FIG. 1. The two halves are symmetrically disposed around the line of symmetry 133.

A transverse line 134 is also indicated in FIG. 1. The transverse line 134 divides the diaper 101 into a front part and a rear part, the distance from the line 134 to the front transverse edge 114 of the diaper constituting 40% of the total length of the diaper, and the distance from the line 134 to the rear transverse edge 115 of the diaper 101 constituting 60% of the total length of the diaper 101.

During use of the diaper 101, the area between the transverse line 134 and the rear transverse edge 115 of the diaper 101 functions as a receiving area 135 for faeces.

The diaper 101 comprises a liquid-permeable cover sheet 102 arranged over that surface of the diaper 101 which, during use, is intended to be directed towards the user, a backing sheet 104 arranged over that surface of the diaper which, during use, is intended to be directed away from the user, an absorption body 106 enclosed between the liquid-permeable cover sheet 102 and the backing sheet 104, and also side flaps 103 arranged outside the absorption body 106.

The liquid-permeable cover sheet 102 extends outside the absorption body 106 along the entire periphery of the absorption body 106. The liquid-permeable cover sheet 102 can consist of any material suitable for the purpose. Examples of commonly used liquid-permeable cover materials are non-woven textile materials, perforated plastic films, net made of plastic or textile, and liquid-permeable foam layers. Liquid-permeable cover materials consisting of continuous thin fibres which extend principally in the longitudinal or transverse direction of the article are also found. Laminates consisting of two or more of the abovementioned possible cover materials are also common, as are covers consisting of different materials within different parts of the surface.

Absorbent articles comprising absorption bodies 106 which have particularly great strength and wear resistance can even function without any extra liquid-permeable cover sheet being required on that side of the article which is directed towards the user during use.

The backing sheet 104 also extends outside the absorption body 106 along the entire periphery of the absorption body 106. Normal backing sheets on absorbent articles are usually liquid-impermeable, but other types of backing sheets are also found. The backing sheet 104 can consist of a number of different materials. It is most common for the backing sheet 104 to consist of a thin liquid-tight plastic film, but it is also possible to use other types of liquid-tight material, such as non-woven material which has been made liquid-tight, for example by coating with plastic, liquid-tight foam sheets, liquid-tight glue or the like. The backing sheet 104 can also consist of a vapour-permeable material. Laminates consisting of at least one liquid-tight material are also found. These laminates usually consist of a liquid-tight material which functions as a liquid barrier and a more textile-like material arranged on that side of the article which is oriented away from the user during use, the outside of the article then being more cloth-like.

The liquid-permeable cover sheet 102 and the backing sheet 104 are interconnected outside the absorption body 106 along the entire periphery of the absorption body 106.

The liquid-permeable cover sheet 102 and the backing sheet 104 can be interconnected in a number of different ways. Examples of connection methods are gluing, heat-melting, ultrasonic welding or the like.

Elastic means 105 are arranged outside the absorption body 106 in those parts of the side flaps 103 of the diaper 101 which essentially run in the longitudinal direction of the diaper 101. The elastic means 105 function as leg elastic and serve the purpose of preventing liquid and excrement from leaking out through the side edges 112, 113 running in the longitudinal direction and in this way, together with surrounding sheets, form outer liquid barriers 108. The elastic means 105 consist of one or more elastic threads which have been applied in a stretched state between the liquid-permeable cover sheet 102 and the backing sheet 104, at least in the crotch portion 123 of the diaper 101. The elastic means 105 are connected to the backing sheet 104 and the cover sheet 102 by gluing, ultrasonic welding or the like.

In alternative embodiments, the elastic means can be arranged on that side of the side flaps 103 which is intended to be directed towards the user during use, or on the opposite side of the side flaps, and are then of course connected only to the cover sheet 102 or the backing sheet 104.

In alternative embodiments, the elastic means can consist of elastic band material made of, for example, foamed material.

The hourglass-shaped absorption body 106 can be constructed from one or more sheets of cellulose fluff pulp. The cellulose fluff pulp can in this connection be mixed with fibres or particles of a highly absorbent polymer material of the kind which chemically binds large quantities of liquid during absorption, to form a liquid-containing gel. The absorption body 106 can also comprise highly absorbent polymer material arranged in a layer inside the absorption body or near the surface or surfaces of the absorption body. The absorption body 106 can also include additional components for improving the characteristics of the absorption body 106. Examples of such components are binding fibres, various types of liquid-spreading sheets or fibres, shape-stabilizing components, reinforcing fibres or the like. The absorption body 106 can of course also consist of other types of absorption material, such as absorbent non-woven material, absorbent foam, textile materials, peat or mixtures of different types of absorption material.

Special sheets for rapidly receiving large quantities of liquid and temporarily retaining this liquid in order then to pass the temporarily stored liquid on to other parts of the absorption body 106 can also be included in diapers of the specified type. Such receiving layers are normally arranged between the liquid-permeable cover sheet 102 and absorption body 106 of the diaper 101. No receiving layer is shown in the figures.

In order to provide further protection against liquid or faeces leaking out over the side edges 112,113 of the diaper 101, said diaper 101 is provided with inner side leakage barriers 109 on that side which, during use, is intended to be directed towards the user. The inner side leakage barriers 109 are arranged adjacent to the longitudinal edges 110 of the absorption body 106 and extend essentially in the longitudinal direction of the diaper 101. Each inner side leakage barrier 109 is made from a separate material strip 111 which has two essentially parallel longitudinal edges 116,117. The material strip 111 is double-folded, the longitudinal edges 116,117 of the material strip 111 then being arranged next to one another. The edges 116, 117 of the material strip 111 are fixed to the cover sheet 102 and constitute the fixed edge of the side leakage barrier. The folded edge of the material strip 111 constitutes the free edge of the side leakage barrier 109.

In the front end portion 121 and the rear end portion 122 of the diaper 101, the inner side leakage barriers 109 are folded down and connected to the cover sheet 102.

The inner side leakage barriers 109 comprise elastic elements 124 connected to the inner side leakage barriers 109 in a pretensioned state. The elastic elements 124 are preferably arranged close to the free edges of the inner side leakage barriers 109. When the pretensioned elastic elements 124 are released, they contract together with the free edges of the inner side leakage barriers 109, the inner side leakage barriers 109 then being brought into a raised configuration away from the liquid-permeable cover sheet 102, at least in the crotch portion 123 of the diaper 101, where the side leakage barriers 109 are not folded down and connected to the cover sheet 102.

The rear and/or front portions of the diaper 101 can also be provided with what is known as waist elastic 125 which consists of elastic means arranged along the front transverse edge 114 and/or the rear transverse edge 115 of the diaper 101 in order to give the diaper 101 a soft and flexible fit around the waist of the user. In this illustrative embodiment, only the rear end portion 122 of the diaper 101 is provided with waist elastic 125 in the form of a thin strip of an elastic foamed material which is attached by glue between the backing sheet 104 and the liquid-permeable cover sheet 102. The waist elastic 125 is applied in a stretched state between the sheets 102, 104 in order to bring about a holding force which stretches the diaper 101 around the waist of the user.

On the rear end portion 122, two soft and inelastic fastening tabs 126 are arranged for securing the diaper 101 around a user, one fastening tab 126 then being arranged on each side portion of the rear end portion 122. During use, the fastening tabs 126 connect the rear end portion 122 to the front end portion 121 by virtue of the fastening tabs 126 having fixing means 127 which can attach to a receiving part arranged on the front end portion 121 of the diaper 101. The fastening tabs 126 are suitably made of a very soft and inelastic material, for example a single non-woven layer or a laminate.

In alternative embodiments, the fastening tabs can be elastic.

The fixing means 127 preferably consists of a male part of a hook-and-loop material and is attached to the fastening tab 126 by, for example, glue on that side of the fastening tab 126 which is directed towards the receiving part during use of the diaper 101.

The receiving part, which is not shown in FIG. 1, for the fastening tab 126 consists of a strip of a receiving material adapted to the fixing means 127 of the fastening tab 126 and extends substantially parallel to the front transverse edge 114 on that side of the diaper directed away from the user during use, that is to say on that side of the backing sheet 104 which is oriented away from the absorption body 106. In the present illustrative embodiment, the receiving material consists of a female part of a hook-and-loop material and is suitably designed so that its extent in the longitudinal direction of the diaper 101 coincides with the width 129 of the fastening tabs 126.

When the diaper 101 is fitted on an infant, the diaper 101 is placed between the infant's legs at the crotch area. The diaper 101 is then closed around the child's waist by virtue of the fastening tabs 126 being made to overlap the front end portion 121 so that the fixing means 127 of the fastening tabs 126 can be applied to the receiving part for securing the diaper.

The fastening tabs 126 are connected to the rear end portion 122 in connection areas 130 which are positioned in those areas of the rear end portion 122 which lie at the side edges 112,113 running in the longitudinal direction. The connection areas 130 consist of parts of the fastening tabs 126 and the parts of the rear end portion 122 which are interconnected. In alternative embodiments, the fixing means 127 of the fastening tabs 126 can consist of pressure-sensitive glue, the receiving part (not shown in FIG. 1) then consisting of a material to which the selected pressure-sensitive glue of the fixing means 127 can be connected so that a suitable bond strength is obtained. Material combinations are usually selected so that the connection between the fixing means 127 and the receiving part can be opened and reclosed for inspecting the diaper 101 during use.

When the diaper 101 is to be changed, the fastening tabs 126 are released from the front end portion 121 of the diaper 101, after which the diaper 101 is opened by removing the front end portion 121 from the child's front.

If the diaper 101 is found to contain faeces, it is common for the parent changing the diaper to try to collect as much of the faeces as possible from the child's skin in the diaper 101 before the latter is folded up and disposed of. The person changing the diaper 101 in this case uses the diaper 101 as a wiping aid, by choosing an area of the diaper 101 which is not soiled with faeces and using this area for the wiping operation. Since the liquid-permeable cover sheet 102 of the diaper 101 is usually not contaminated with faeces in the front end portion 121, it is in most cases this area which is used in the wiping operation.

The diaper 101 according to the invention is characterized principally in that it has an area 131 specially arranged and adapted for wiping faeces from the skin of a child soiled with faeces. The area 131 in this case comprises a material section 132 which constitutes a wiping material 136, specially adapted for wiping and removing faeces from a child's skin.

The material section 132 is arranged in the front end portion 121 of the diaper 101, by being applied on that side of the liquid-permeable cover sheet 102 which, during use, is intended to bear against the user's skin. Alternative locations of the material section 132, in areas of the diaper 101 which are not normally soiled with faeces, are also conceivable. The front transverse edge 137 of the material section 132 is arranged at the front transverse edge 114 of the diaper 101, but in alternative embodiments it can be arranged slightly away from the front transverse edge 114 of the diaper 101. The material section 132 also has a rear transverse edge 138 located at the boundary between the front end portion 121 of the diaper 101 and the crotch portion 123. In alternative embodiments, the rear transverse edge 138 of the material section 132 can be located nearer to the rear transverse edge 114 of the diaper 101. In further embodiments, the material section 132 can extend a short distance into the crotch portion 123, the rear transverse edge 138 of the material section 132 then being arranged in the crotch portion 123.

To ensure that the material section 132 will function as a wiping material 136, the material section 132 must have a certain minimum surface area, namely a minimum extent in the longitudinal direction of the diaper 101 and a minimum extent in the transverse direction of the diaper 101. The surface area must be of such a size that it is sufficient to accommodate the amount of faeces the diaper 101 is expected to be able to hold. This amount differs of course depending on whether the diaper is intended for younger or older children, or for adults. The surface area must also be of such a size that it constitutes a physically effective wiping surface when the diaper 101 is crumpled up and gripped during wiping. On the other hand, the material section 132 should not be so great that it extends into the central receiving area of the diaper 101 provided for urine, since specific urine-receiving properties are not easy to combine with specific wiping properties.

it has been found that the material section 132 should have a minimum extent of 10% of the total length of the diaper 101 In order to be able to accommodate the expected amounts of faeces and in order to constitute a physically effective wiping surface. However, in order to avoid interference with the urine-receiving area of the diaper 101, it is expedient that the material section 132 does not extend more than 40% of the total length of the diaper 101 from the front transverse edge 114 of the diaper 101, in the longitudinal direction of said diaper 101. This means that the material section 132 should not extend beyond the transverse line 134 indicated in FIG. 1.

Regardless of whether the diaper 101 is intended for newborn babies with a body weight of 2-4 kilograms or the diaper 101 is intended for bigger children with a body weight of up to about 25 kilograms, it has been found that the abovementioned limits are valid. The most suitable extent of the material section 132 in the longitudinal direction of the diaper 101 is 25-35% of the total length of the diaper 101. With such an extent in the longitudinal direction of the diaper 101, a wiping surface is obtained which in principle is always sufficiently large in terms of the amount of faeces to be collected. The surface area of the material section 132 is also such that it constitutes a sufficiently large wiping surface when the diaper 101 is gripped/crumpled up during the wiping operation. A material section 132 with this extent in the longitudinal direction of the diaper can also be arranged outside the urine-receiving area of the diaper 101, by a good margin, so that the properties of the diaper in respect of receiving urine are not compromised.

The material section 132 is arranged symmetrically about the longitudinal line of symmetry of the diaper 101, the material section 132 extending across the full width of the diaper 101. The material section 132 has the same shape as the liquid-permeable cover sheet 102 and backing sheet 104 of the diaper 101 in the front end portion, said material section 132 covering the whole of the front portion 121. Alternatively, the material section can have an extent in the transverse direction of the diaper 101 which is smaller than the extent of the diaper 101 in the transverse direction at the front portion 121. However, the material section should have an extent of at least 12 cm, preferably of at least 15 cm, in the transverse direction.

The material section 132 is connected to the liquid-permeable cover sheet 102 by gluing, hot melting, ultrasonic welding or a similar suitable method. The connection can be made across the entire surface of the material section 132 or along two or more of the edges of the material section 132. The connection can be made up of a number of discrete binding elements distributed substantially uniformly across the material section 132, or by a continuous connection across the whole surface of the material section 132. The discrete binding elements can have a circular shape, a star shape or the shape of oblong rectangles.

It is also possible to arrange connections in the form of continuous connection lines across the surface of the material section 132, in which case the continuous connection lines expediently extend in two substantially perpendicular directions in relation to one another.

In the present illustrative embodiment, the inner side leakage barriers 109 of the diaper 101 are arranged across the material section 132, that is to say on that side of the material section 132 which is oriented towards the user during use.

In alternative embodiments, the material section 132 can have an extent, in the transverse direction of the diaper 101, which is smaller than or equal to the distance between the two inner side leakage barriers 109 of the diaper, the material section 132 being arranged completely between the inner side leakage barriers 109.

It is also conceivable to form the diaper 101 such that the inner side leakage barriers 109 do not extend all the way to the front transverse edge 114, in which case the material section 132 and the inner side leakage barriers 109 do not necessarily need to interfere with one another.

In another embodiment, the material section 132 can be folded together with the inner side leakage barriers 109 and thus constitute parts of the respective side leakage barrier 109.

Since the area 131 consisting of the special wiping material 136 does not interfere with the urine-receiving area of the diaper 101, the demands in respect of letting liquid pass through, and other urine-related properties, are not as great in the area 131 as in the urine-receiving area of the diaper 101. The urine-receiving area of the diaper 101 consists essentially of the crotch portion 123. This means that certain urine-related properties can be waived in favour of wiping-related properties for the wiping material 136.

A great deal of information on how a material adapted for removing/wiping faeces from the skin should be formed is available in the literature and from specialists in the field of wiping materials/toilet paper.

A wiping material 136 applied on the liquid-permeable cover sheet 102 of a diaper 101 according to the present invention must have most of the properties of toilet paper, and other properties besides.

A property which is needed over and above the normal properties of toilet paper is that the wiping material 136 be smooth and strong so that it can bear against a user's body during the time a diaper 101 is worn, without the skin being affected in an adverse way or the wiping properties of the wiping material 136 deteriorating appreciably. Smoothness is a property closely associated with friction between the material and the user's skin, for which reason low friction against the skin is a parameter which is of importance for the wiping material 136 both when the wiping material 136 is dry and when it Is wet, and when the user's skin is wet or dry.

A baby diaper 101 is normally used for 3-8 hours during the day and for up to 10-12 hours during the night, so that it may be wet during much of the time it is in use, especially at night time.

Nor should the wiping material 136 leave residues of fluff on the infant's skin during long periods of use, even if it is moist for a large part of the time of use.

Of course, the wiping material 136 must not cause any allergic reactions or skin irritation despite bearing against the body for a long period in the dry or wet state.

Properties which the wiping material 136 has in common with the properties of toilet paper are, above all, the adsorption capacity of the material and the surface structure of the material.

Adsorption capacity is defined as the ability of a material to bind another material to its surface, and, in connection with the present invention, the ability of the wiping material 136 to bind faeces to its surface is of course of central importance.

The surface structure of the wiping material 136 is of great importance for how well the material is able to quickly take up faeces during wiping. The surface structure also determines, on the one hand, how well faeces attaches to the material and, on the other hand, how well faeces can be stored on the material.

The wiping material 136 has a folded three-dimensional structure comprising elevations and depressions. The material will in this case have a frequency of elevations and depressions of 1-20 cycles/cm, preferably 2-12 cycles/cm, and more preferably 3-8 cycles/cm. The term “frequency” here signifies the number of elevations which the material includes along a distance of 1 cm from any point on the material in the direction having the highest number of elevations from said point. When determining the frequency of elevations and depressions on a wiping material 136, the frequency is measured from at least ten substantially uniformly distributed points across the wiping material 136, the frequency being calculated as a mean value of measured frequencies.

The three-dimensional surface structure of the wiping material 136 must be at the macroscopic level, that is to say the three-dimensional structure must be observable by sight at normal reading distance. The surface structure must also have a certain resilience and stability so that it does not collapse in connection with the wiping operation. Such a surface structure is able to catch faeces during the wiping operation and also store faeces in its folds and depressions.

In an alternative embodiment, the wiping material 136 specially adapted for wiping/removing faeces from a child's skin can consist of a material comprising openings which are able to collect faeces during the wiping operation and thus help by catching/storing the faeces in the openings. The openings will in this case have a frequency of 1-20 openings/cm, preferably 2-12 openings/cm, and most preferably 3-8 openings/cm. The frequency is measured analogously to the frequency of elevations and depressions in a material according to the above. For a wiping material comprising openings, the frequency is once again calculated as a mean value of ten measurements distributed substantially uniformly across the material section 132.

The folded wiping material 136 comprising elevations and depressions has been folded in the production process, after which it has been connected in the folded state to the liquid-permeable cover sheet 102 of the diaper 101. The folding has been generated by passing the wiping material 136 through a roller in the production apparatus which comprises radially oriented elevations and depressions. The roller also comprises means for generating an underpressure so that the wiping material 136 has been sucked against the roller and has been shaped according to the elevations and depressions of the roller. Finally, the wiping material 136 has been connected to the liquid-permeable cover sheet 102 of the diaper before being removed from the roller.

Alternatively, the wiping material can be folded by connecting pre-tensioned elastic means to the wiping material, in which case folds form as the elastic means contract when the pre-tensioning forces are released.

Another way of producing a folded wiping material is to prefabricate a laminate consisting of a plane material and a folded material, in which case the laminate can be produced according to any of the methods described above. The folded material has in this case been locked, in the folded state, to the plane side of the laminate. Patent EP 673,314 describes a method of producing a folded laminate in accordance with the above.

Creped material can also be used for the material section 132.

The wiping material 136 is expediently a fibrous material comprising cellulose fibres such as paper pulp fibres, cotton fibres or the like. Synthetic fibres such as rayon fibres, polyethylene fibres or polypropylene fibres can expediently be mixed with the cellulose fibres to increase the strength and wear resistance of the wiping material 136.

Because of the requirement for smoothness and softness, cellulose fibres produced by the chemical process are preferable to mechanically produced cellulose fibres. Cellulose fibres produced by the chemical process have a smaller diameter than cellulose fibres produced by the mechanical process, chemically produced cellulose fibres being slimmer than cellulose fibres produced by the mechanical process. A wiping material 136 comprising chemically produced cellulose fibres therefore has greater softness and smoothness against the user's skin.

An example of a suitable wiping material 136 is described in U.S. Pat. No. 4,902,564, where the wiping material 136 comprises a mixture of synthetic fibres and cellulose fibres. According to the patent, the wiping material 136 can contain 50-75% cellulose fibres and 25-50% polyester fibres. To give the material a high degree of strength against wear, the fibres are entangled, the longer synthetic fibres acting as an effective reinforcement of the short cellulose fibres. The material is also to a large extent lint-free. To further increase the strength of the wiping material 136, an adhesive binder can also be added. After entangling and drying, the wiping material 136 can also be textured or creped in order to increase the material's softness, bulk and textile feel.

Another example of a material which is suitable as wiping material 136 is described in U.S. Pat. No. 4,906,513. The patent describes a non-woven material of laminate structure with a relatively heavy middle layer consisting of thermoplastic microfibres produced by a meltblown process and added fibres. On one side, the laminate has a layer of low basis weight comprising substantially continuous thermoplastic filaments of large diameter. On the other side, the laminate structure has a microfibre layer. The described material is said to be strong, have a cloth feel, and be usable for a number of different applications. According to U.S. Pat. No. 4,906,513, the surface layers give a laminate with high strength and a low tendency to fluffing. The combination of different layers gives the material good wiping properties. The laminate is preferably bound together by application of heat and pressure, and the individual components are preferably treated with a surface-active agent to improve the wettability of the material. A preferred combination consists of a middle layer of microfibres of meltblown polypropylene with added fibres suitably consisting of cellulose fibres, and with a surface layer of spunbond polypropylene, and with the other surface layer consisting of microfibres which can be filaments of meltblown polypropylene. Examples of suitable wetting agents are Aerosol TO, Triton X-100 and Triton X0102.

In addition to reinforcement by means of synthetic fibres, it is possible to add chemicals to increase the strength of the wiping material 136. Regarding the strength of the wiping material, it is chiefly the wet strength of the material that has to be considered, the dry strength seldom causing any problem. Polyaminoamide-epichiorohydrin (PME) is an example of a commercially available chemical for increasing the strength of a wiping material 136. Another possible chemical is glyoxylated polyacrylamide (G-PAM).

Admixture of microfibres, at least on the surface of the wiping material 136 directed towards the body, increases the softness and smoothness of the wiping material 136 as the thin fibres are both soft and slender. The wear resistance of the material and its wet strength are also affected positively by use of microfibres.

For the thin fibres or filaments to provide the properties which are required of a microfibre material intended as wiping material 136, the fibres must have a coarseness of not more than 1 denier, preferably less than 0.5 denier. For circular cross sections, and of course depending on the density of the polymer, 1 denier corresponds to a fibre diameter of the order of 10-11 micrometres, while 0.5 denier corresponds to approximately 7-8 micrometres.

There are also chemicals/agents which increase the softness and smoothness of the wiping material 136. An agent for increasing softness and smoothness can, in physical form, consist of a solution, suspension, cream, lotion, ointment, paste, gel, foam, aerosol, or be present in solid phase as particles, flakes, fibres, films, foam, wadding, sticks, etc. Cream, lotion or ointment is preferably suitable for the wiping material 136, but others of the above-described forms are of course also conceivable. Agents for increasing the softness and smoothness of the wiping material 136 can, for example, include lipids (fats, oils, waxes), solvents (including water), water-soluble substances, surface-active agents (emulsifiers, surfactants), viscosity regulators, pH regulators, preservatives, complexing agents (e.g. chelates). The lipids are usually emulsified in water, so-called oil-in-water emulsion, or water is emulsified in the lipid phase, so-called water-in-oil emulsion. Swedish patent application SE 520,236 describes other skin-care agents suitable for the wiping material 136.

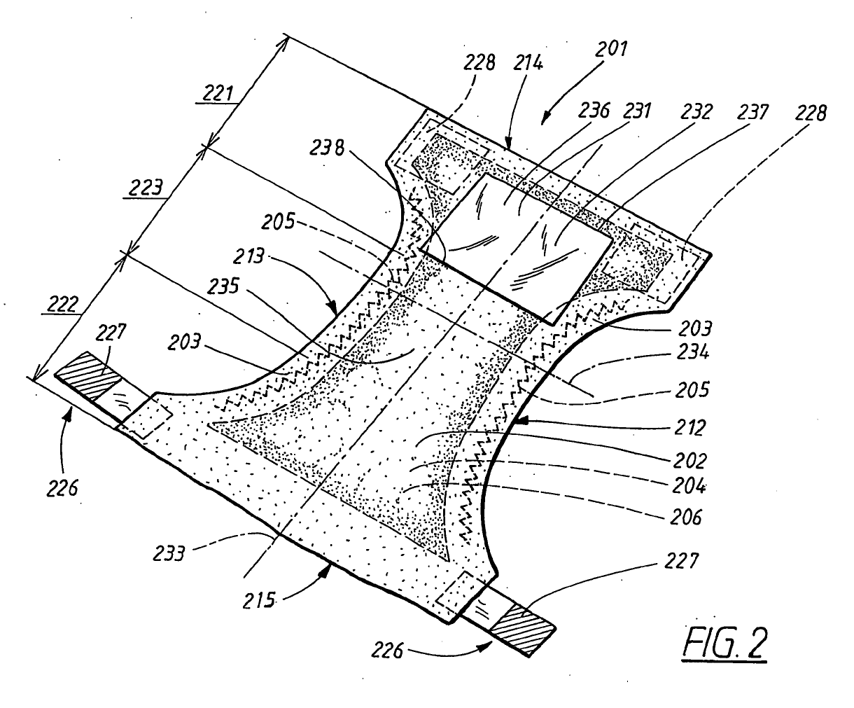

FIG. 2 shows an incontinence guard 201 intended to be used by an adult suffering from incontinence. The incontinence guard 201 is shown from that side which is intended to be directed towards the user during use, the incontinence guard 201 being shown stretched flat out.

Reference numbers used in FIG. 2 and which correspond to FIG. 1 have the same reference in the number series 200-299. For example, the front end portion has reference number 121 in FIGS. 1 and 221 in FIG. 2.

The incontinence guard 201 is essentially hourglass-shaped and has longitudinal edges 212, 213, a front transverse edge 214 and a rear transverse edge 215. The incontinence guard 201 also has a front end portion 221, a rear end portion 222 and a narrower crotch portion 223 located between the end portions 221, 222.

The incontinence guard 201 comprises a liquid-permeable cover sheet 202, arranged across that surface of the incontinence guard 201 which, during use, is intended to be directed towards the user, a backing sheet 204 arranged across that surface of the incontinence guard 201 which, during use, is intended to be directed away from the user, and an absorption body 206 enclosed between the liquid-permeable cover sheet 202 and the backing sheet 204.

A longitudinal line of symmetry 233 which divides the incontinence guard 201 into a right-hand half and a left-hand half is indicated in FIG. 2, the two halves being symmetrically disposed around the line of symmetry 233.

A transverse line 234 Is also indicated in FIG. 2. The transverse line 234 divides the incontinence guard 201 into a front part and a rear part, the distance from the line 234 to the front transverse edge 214 of the incontinence guard 201 constituting 40% of the total length of the

Incontinence guard 201, and the distance from the line 234 to the rear transverse edge 215 of the incontinence guard 201 constituting 60% of the total length of the incontinence guard 201.

During use of the incontinence guard 201, the area behind the transverse line 234 functions as a receiving area 235 for faeces.

Elastic means 205 are arranged outside the absorption body 206 in those parts of the side flaps 203 of the incontinence guard 201 which essentially run in the longitudinal direction of the incontinence guard 201. The elastic means 205 function as leg elastic and serve the purpose of preventing liquid and excrement from leaking out through the side edges 212, 213 running in the longitudinal direction.

At the rear end portion 222, two fastening tabs 226 are arranged for securing the incontinence guard 201 around a user. During use, the fastening tabs 226 connect the rear end portion 222 to the front end portion 221 by virtue of the fixing members 227 of the fastening tabs 226 being fixed to special receiving surfaces 228 arranged on the backing sheet 204 of the incontinence guard 201.

The incontinence guard 201 has an area 231 specially arranged for wiping/removing faeces from soiled skin. The area 231 in this case comprises a material section 232 consisting of a special wiping material 236 adapted for wiping/removing faeces from the skin of an incontinent user.

The wiping material 236 is arranged in the front end portion 221 of the incontinence guard 201 and is applied on that side of the liquid-permeable cover sheet 202 which, during use, is intended to bear against the user.

To keep down the costs of the material section 232, the material section 232 does not extend all the way to the front transverse edge 214 of the incontinence guard 201, the front transverse edge 237 of the material section 232 being arranged at a distance from the front transverse edge 214 of the incontinence guard 201. This is possible because the front part of the incontinence guard 201, that is to say the part not constituting the receiving area 235 for faeces, has such a large surface that there is no problem in creating a sufficiently large area 231 specially arranged for wiping/removing faeces.

To ensure that the material section 232 will function as a wiping material 236, the material section 232 must have a certain minimum surface area, namely an extent in the longitudinal direction of the incontinence guard 201 and an extent in the transverse direction of the incontinence guard 201. The surface area must be of such a size that it is sufficient to accommodate the amount of faeces the incontinence guard 201 is expected to be able to hold.

On the other hand, the wiping material 232 should not be so great that it extends into the central receiving area of the incontinence guard provided for urine, since urine-receiving properties are not easy to combine with wiping properties.

For the incontinence guard 201, it has been found that the material section 232 should have a minimum extent, in the longitudinal direction of the incontinence guard 201, of 20% of the total length of the incontinence guard 201 in order to be able to accommodate the expected amounts of faeces from an incontinence guard 201 of specific size.

In order to avoid interference with the urine-receiving area of the incontinence guard 201, it is expedient that the material section 232 does not extend more than 30% of the total length of the incontinence guard 201 from the front transverse edge 214 of the incontinence guard 201, in the longitudinal direction of said incontinence guard 201.

Regardless of whether the incontinence guard 201 is intended for incontinent persons of small stature, in which case the incontinence guard 201 has a total length of less than 70 cm, or is intended for incontinent persons of larger stature and has a length in excess of 90 cm, it has been found that certain limits apply.

The most suitable extent of the material section 232 in the longitudinal direction of the incontinence guard is 25% of the total length of the diaper 101. With this extent of the material section 232, in the longitudinal direction of the incontinence guard 201, a wiping surface is obtained which in principle is always sufficiently large in terms of the amount of faeces to be collected. A material section 232 with this extent in the longitudinal direction of the diaper can also be arranged outside the urine-receiving area of the incontinence guard 201, by a good margin, so that the properties of the diaper in respect of receiving urine are not compromised. The material section 232 according to the embodiment in FIG. 2 has a rear transverse edge 238 arranged well outside the urine-receiving area of the incontinence guard 201.

The material section 232, according to the present embodiment, is arranged symmetrically about the longitudinal line of symmetry of the incontinence guard 201, the material section 232 having a width of 20 cm in the transverse direction of the incontinence guard 201.

In alternative embodiments, it is conceivable for the material section to have an extent of at least 15 cm, preferably of at least 18 cm, in the transverse direction of the diaper 101. It is also possible to let the material section 232 extend across the full width of the incontinence guard 201 in the same way as is shown for a baby diaper 101 in FIG. 1.

The material section 232 is expediently connected to the liquid-permeable cover sheet 202 by gluing, hot melting, ultrasonic welding or a similar suitable method. The connection can be made across the entire surface of the material section 232 or along two or more of the edges of the material section 232.

In terms of choice of material, the material section 232 is constructed in the same way as the corresponding material section 132 provided for wiping/removing faeces in the diaper 101 described above.

The invention also includes all conceivable combinations of the described illustrative embodiments.

Moreover, the invention is not limited to the abovementioned illustrative embodiments, and instead it is of course applicable to other embodiments, especially to other types of absorbent articles within the scope of the attached patent claims.

1. An absorbent article, comprising a liquid-permeable cover sheet intended to be directed towards a user during use, a backing sheet intended to be directed away from a user during use, an absorbent body arranged between the liquid-permeable cover sheet and the backing sheet, a rear transverse edge, a front transverse edge, a distance between the rear transverse edge and the front transverse edge constituting a total length of the absorbent article, the absorbent article comprising a receiving area for faeces arranged on the liquid-permeable cover sheet between a transverse line, located at a distance of about 60% of the total length of the article away from the rear transverse edge and the rear transverse edge of the absorbent article, the absorbent article further comprising a wiping area intended for removing faeces, said wiping area being arranged outside the receiving area for faeces, the wiping area comprises a wiping material connected to the absorbent article.

2. The absorbent article according to claim 1, wherein the wiping material is arranged on the liquid-permeable cover sheet.

3. The absorbent article according to claim 1, wherein the absorbent article is a diaper for infants, in which the wiping material has an extent, in the longitudinal direction of the diaper, of 10-40% of the total length of the diaper.

4. The absorbent article according to claim 3, wherein the wiping material has an extent, in the transverse direction of the diaper, of at least 12 centimetres.

5. The absorbent article according to claim 3, wherein the wiping material has an extent across the full width of the diaper in the transverse direction of said diaper.

6. The absorbent article according to claim 1, wherein the absorbent article; is an incontinence guard for adults, in which the wiping material has an extent, in the longitudinal direction of the incontinence guard, of 20-30%, of the total length of the incontinence guard.

7. The absorbent article according to claim 6, wherein the wiping material has an extent, in the transverse direction of the incontinence guard, of at least 15 centimetres.

8. The absorbent article according to claim 6, wherein the wiping material has an extent across the full width of the incontinence guard in the transverse direction of said incontinence guard.

9. The absorbent article according to claim 1, wherein the wiping material comprises cellulose fibres.

10. The absorbent article according to claim 1, wherein the wiping material comprises rayon fibres.

11. The absorbent article according to claim 1, wherein the wiping material comprises microfibres.

12. The absorbent article according to claim 9, wherein the wiping material comprises means which increase the wet strength of the wiping material.

13. The absorbent article according to claim 9 wherein the wiping material comprises means which increase the softness of the wiping material.

14. The absorbent article according to claim 1, wherein the wiping material has a folded three-dimensional structure.

15. The absorbent article according to claim 14, wherein the folding of the wiping material has a frequency 1-20 cycles/centimeter.

16. The absorbent article according to claim 1, wherein the wiping material has a structure comprising openings.

17. The absorbent article according to claim 16, wherein the openings of the wiping material have a frequency of 1-20 openings/centimetre.

18. The absorbent article according to claim 1, wherein the absorbent article is a pair of diaper pants.

19. The absorbent article according to claim 1, wherein the absorbent article is a belt diaper.

20. The absorbent article according to claim 1, wherein the wiping material is arranged on the backing sheet of the absorbent article.

21. The absorbent article according to claim 1, wherein the absorbent article is a diaper for infants, in which the wiping material has an extent, in the longitudinal direction of the diaper, of 25-30% of the total length of the diaper.

22. The absorbent article according to claim 1, wherein the wiping material is softer than the receiving area of the liquid-permeable cover sheet, wherein softness is inversely proportional to the friction between the material and the user's skin when the material and the skin are wet.

23. The absorbent article according to claim 3, wherein the wiping material has an extent, in the transverse direction of the diaper, of at least 15 centimetres.

24. The absorbent article according to claim 1, wherein the absorbent article is an incontinence guard for adults, in which the wiping material has an extent, in the longitudinal direction of the incontinence guard, of 25% of the total length of the incontinence guard.

25. The absorbent article according to claim 6, wherein the wiping material has an extent, in the transverse direction of the incontinence guard, of at least 18 centimetres.

26. The absorbent article according to claim 14, wherein the folding of the wiping material has a frequency 3-8 cycles/centimeter.

27. The absorbent article according to claim 16, wherein the openings of the wiping material have a frequency of 3-8 openings/centimetre.
